(19) United States
(12) Patent Application Publication
(10) Pub. No.: US 2011/0184497 A1
(43) Pub. Date: Jul. 28, 2011

(54) AC LOGIC POWERED STIMULATOR IC FOR NEURAL PROSTHESIS

(75) Inventors: Sung June Kim, Seoul (KR); Choong Jae Lee, Seoul (JP); Tae Ho Jeon, Gyeonggi-do (JP); Soon Kwan An, Seoul (JP); William J. Heetderks, Silver Spring, MD (US)

(73) Assignees: SNU R&DB FOUNDATION, Seoul (KR); NURO BIO-SYSTEMS CORP., Suwon-si, Gyeonggi-do (KR)

(21) Appl. No.: 13/003,800

(22) PCT Filed: Jul. 14, 2009
(86) PCT No.: PCT/KR09/03872

§ 371 Date: Mar. 30, 2011

(30) Foreign Application Priority Data
Jul. 14, 2008 (KR) 10-2008-0068248

(51) Int. Cl. A61N 1/375; A61N 1/36

(52) U.S. Cl. 607/72; 607/2

(57) ABSTRACT

Disclosed is a neural stimulator for neural prosthesis: including a power receiver which receives power from outside and supplies the received power to a circuitry including an electrical signal generator; the electrical signal generator which generates a neural stimulating electrical signal; and a casing which protects the power receiver and the electrical signal generator from bodily fluid, wherein the power supplied by the power receiver to the circuitry including the electrical signal generator is AC logic power. In accordance with this disclosure, a neural stimulator with reduced production cost, simplified production process and reduced size, as compared with those of the related art, while being safe, may be provided.

### TECHNICAL FIELD

This disclosure relates to a neural stimulator for neural prosthesis, more particularly to a neural stimulator of which being simply coated, without using a hermetic casing, to reduce production cost and using AC logic power to ensure safety.

### BACKGROUND ART

Neural prostheses are a series of mechanical or electronic artificial devices attached to or implanted in the human body in order to substitute or aid sensory or motor disabilities caused by congenital or acquired neural injuries.

Technologies involved in neural prosthesis include neural stimulators, complete hermetic packaging, neural signal acquisition, neural stimulation electrodes, neural signal processing, biotelemetry, or the like.

Among them, complete hermetic packaging is required for protecting the stimulator or other parts of a neural prosthetic device from biological fluids or ions. And the size of packaging is also an important factor to consider in designing for easier implantation into the body and absence of inconvenience in daily lives. Moreover, the complete hermetic packaging requires high cost and complicated processes for the hermetic sealing to protect the stimulator or other parts. The production cost of the neural prosthetic device increases with the cost for the packaging. However, since most of the people who need neural prosthetic devices are those who cannot participate in economic activities, they find it difficult to get help from the expensive neural prosthetic devices. Accordingly, the cost for the packaging of the neural prosthetic device needs to be reduced.

In addition, since the prosthetic device is inserted into the human body, the followings have to be considered when designing the device or system for neural prosthesis. Since they are exposed to the bodily fluids or ions for a long time, the device or system needs to have good reliability and safety, being unharmful to the human tissues.

The reliability means that the device or system for neural prosthesis operates stably for the expected period of time without corrosion in the human body. This may be attained by hermetically sealing the circuitry, device or system inserted in the body with a casing. However, as described earlier, the hermetic casing is disadvantageous in cost and processes.

And, the safety means that, when inserted into or attached on the human body, the neural stimulator gives no harm to the human body. To be unharmful to human tissues, the circuitry needs not to produce toxic substances or cause irreversible reactions in the human body. The irreversible reaction refers to a chemical reaction which results in necrosis of nearby tissues or loss of electrochemical equilibrium in the human body.

Accordingly, a neural prosthetic device provided with reliability and safety as well as economy is required.

### DISCLOSURE OF INVENTION

1. Technical Problem

This disclosure is directed to providing a neural stimulator for neural prosthesis, the outside of which being coated by a circuitry protecting material instead of using complete hermetic packaging. The disclosure is also directed to providing a neural stimulator using AC logic power instead of DC power in order to safely protect cells or tissues.

2. Technical Solution

In an aspect, there is provided a neural stimulator for neural prosthesis including: a power receiver which receives power from outside and supplies the received power to a circuitry including an electrical signal generator; the electrical signal generator which generates a neural stimulating electrical signal; and a casing which protects the power receiver and the electrical signal generator from bodily fluid, wherein the power supplied by the power receiver to the circuitry including the electrical signal generator is AC logic power.

In another aspect, there is provided a neural stimulator for neural prosthesis including: a power receiver which receives power from outside and supplies the received power to a power converter; an electrical signal generator which generates a neural stimulating electrical signal; the power converter which converts the power received from the power receiver into stable AC logic power and supplies it to a circuitry including the electrical signal generator; and a casing which protects the power receiver, the electrical signal generator and the power converter from bodily fluid, wherein the power supplied by the power converter to the circuitry including the electrical signal generator is AC logic power.

### ADVANTAGEOUS EFFECTS

In accordance with this disclosure, the outside of the neural stimulator for neural prosthesis is coated with a circuitry protecting material instead of complete hermetic packaging.

Further, AC logic power is used instead of DC power to safely protect cells or tissues.

Accordingly, a neural stimulator with reduced production cost, simplified production process and reduced size, as compared with those of the related art, while being safe, may be provided.

Further, AC logic power is used to safely protect cells or tissues in case of current leakage even when hermetic packaging fails.

### BRIEF DESCRIPTION OF DRAWINGS

The above and other aspects, features and advantages of the disclosed exemplary embodiments will be more apparent to the following detailed description taken in conjunction with the accompanying drawings in which:

FIG. 1 is a schematic diagram illustrating a neural stimulator for neural prosthesis according to an embodiment;

FIG. 2 shows damage of tissues by monophasic or biphasic waveforms;

FIG. 3 is a circuit diagram of a double layer modeled as an electrical circuitry;

FIG. 4 is a circuit diagram illustrating a simulation of a double layer modeled as an electrical circuitry;

FIG. 5 shows frequency response of the voltage applied between both ends of the capacitor in FIG. 4; and

FIG. 6 is a schematic diagram illustrating a neural stimulator for neural prosthesis according to another embodiment to which a power converter is equipped.

### MODE FOR THE INVENTION

Exemplary embodiments now will be described more fully hereinafter concerning the accompanying drawings, in which exemplary embodiments are shown. This disclosure may, however, be embodied in many different forms and should not be construed as limited to the exemplary embodiments set forth therein. Rather, these exemplary embodiments are provided so that this disclosure will be thorough and complete, and will fully convey the scope of this disclosure to those skilled in the art. In the description, details of well-known features and techniques may be omitted to avoid unnecessarily obscuring the presented embodiments.

The terminology used herein is for describing particular embodiments only and is not intended to be limiting of this disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item. The use of the terms “first”, “second” and the like does not imply any particular order, but they are included to identify individual elements. Moreover, the use of the terms first, second, etc. does not denote any order or importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

In the drawings, like reference numerals in the drawings denote like elements. The shape, size and regions, and the like, of the drawing may be exaggerated for clarity.

The most important factors in designing a neural stimulator for neural prosthesis are efficiency and safety.

The efficiency refers to the ability of eliciting a desired physiological response. Stimulation most similar to the original neural stimulation may be found by controlling the magnitude of stimulation.

The safety is considered in two important aspects. First, the tissues or cells to which the stimulation is applied should not be damaged. Second, the electrode of the stimulator should not be damaged, for example, by corrosion. The safety issue will be considered in the followings.

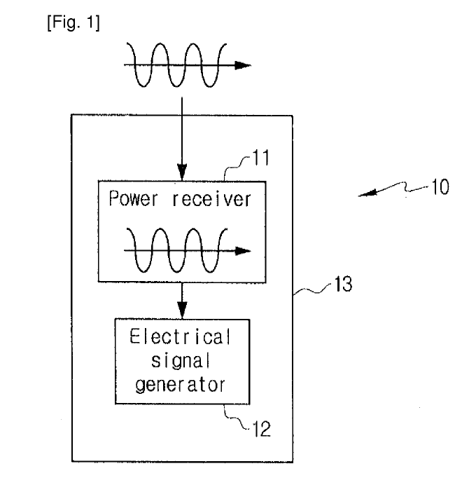

FIG. 1 is a schematic diagram illustrating a neural stimulator for neural prosthesis according to an embodiment. The neural stimulator for neural prosthesis 10 comprises: a power receiver 11 which receives power from outside and supplies the received power to a circuitry including an electrical signal generator; the electrical signal generator 12 which generates a neural stimulating electrical signal; and a casing 13 which protects the power receiver and the electrical signal generator from bodily fluid, wherein the power supplied by the power receiver to the circuitry including the electrical signal generator is AC logic power. The casing coats with a circuitry protecting material the outside of the power receiver and the electrical signal generator.

The power receiver 11 supplies power for driving the neural stimulator for neural prosthesis. The power used by the neural stimulator for neural prosthesis may be received from outside. The power receiver 11 may supply the received power without conversion. Since the neural stimulator for neural prosthesis is implanted into the body, it is restricted in size and volume, and, therefore, there may be a need to construct a circuitry without a power conversion device.

The electrical signal generator 12 generates a neural stimulating electrical signal. The signal generated by the electrical signal generator 12 is produced in the form of electrical current or voltage according to a desired magnitude of stimulation.

The casing 13 protects the circuitry constituting the neural stimulator for neural prosthesis, e.g., the power converter or the electrical signal generator, from bodily fluids or ions. The protection from bodily fluids or ions will be described in detail below.

In general, the outside of a neural prosthetic device including a stimulator chip for neural prosthesis is hermetically sealed to prevent exchange of matter with outside bodily fluids or ions. To this end, the casing is made of a material resistant to corrosion by the bodily fluids or ions. Commonly, titanium or ceramics are used.

There are some problems associated with the hermetic sealing of the casing. The biggest problem is to seal the electrical lead. Since the electrical lead has to be insulated from the casing, a further cost is required for a complicated process of insulating. Accordingly, there is a need that the neural prosthetic device operates safely without a hermetic casing to produce an inexpensive neural prosthetic device.

For this purpose, the casing 13 needs to have a simpler and less expensive structure, rather than a complete hermetic packaging type. A method of coating simply with an insulating material capable of protecting an integrated chip may be considered, although there may be a little leakage. The method of coating simply refers to enclosing the circuitry rather than employing a complete hermetic packaging, so that it may be appropriate for large-scale production. For example, a method of depositing layers in a semiconductor process may be employed.

Especially, silicone elastomer or polyurethane is effective in protecting silicon oxide or metal oxide systems. Since silicone strongly binds to molecules and stabilizes molecular surfaces, it prevents corrosion of silicon oxide or other oxides. Accordingly, the casing 13 may coat with silicone elastomer or polyurethane the circuitry or devices enclosed thereby.

When the outside of the circuitry is coated with a circuitry protecting material without employing a complete hermetic packaging, leakage of power or stimulation signal may occur inside the human body. The leakage of power or stimulation signal inside the human body may result in damage of nearby tissues or cells. Especially, when DC power is used, charge accumulation at the metal tissue interface by monophasic stimulations in nearby tissues or cells may be fatal.

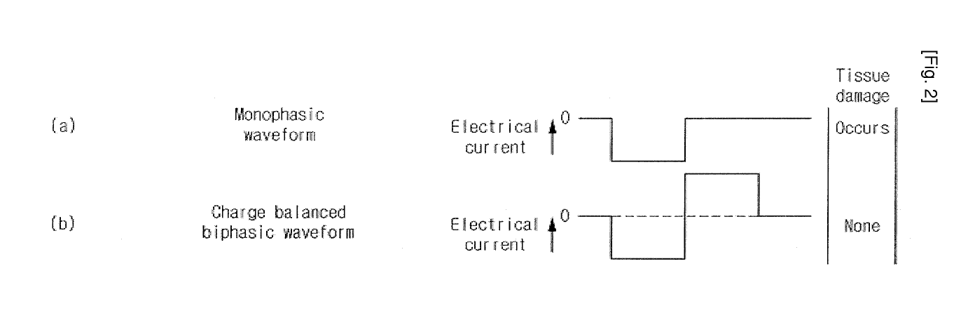

FIG. 2 compares damage of tissues by monophasic or biphasic waveforms. The monophasic waveform damages tissues, whereas the biphasic waveform does not damage tissues.

In the graph, the monophasic waveform may correspond to DC power, and the biphasic waveform may correspond to AC logic power with alternating positive and negative values.

As seen in FIG. 2, use of DC power may be problematic in the safety of tissues and cells, as compared with use of AC logic power. Accordingly, considering that leakage of power may occur because of the lack of complete hermetic packaging, use of AC logic power rather than of DC power may be safer for tissues or cells. Therefore, using AC logic power for a neural stimulator may be worth considering.

Either a nonFaradaic reaction or a faradaic reaction may occur in the charge transfer at the interface between an electrode and an electrolyte. The nonFaradaic reaction refers to a reaction with no transfer of electrons between the electrode and electrolyte, whereas the faradaic reaction refers to a reaction with transfer of electrons between the electrode and electrolyte, thereby resulting in oxidation and reduction. The faradaic reaction is divided into reversible and irreversible reactions.

The irreversible reaction refers to a reaction where a reaction toward one direction predominates and the other hardly occurs. In general, a reaction where gas or precipitate is formed is an irreversible reaction. As the gas or precipitate formed by the reaction of ion or substance exits the reacting system, the reverse reaction hardly occurs. The irreversible reaction leads to a change in chemical environment, thereby resulting in damage of tissues, cells, or the electrode. Therefore, it is important to prevent the occurrence of irreversible reactions when designing a neural stimulator for neural prosthesis.

Typical irreversible reactions occurring at the interface between the electrode and electrolyte are as follows.

MathFigure 1

2H₂O+2e⁻→H₂↑+2OH⁻ (reduction of water) [Math.1]

MathFigure 2

2H₂O→O₂↑+4H⁺+4e⁻ (oxidation of water) [Math.2]

Oxidation or reduction of water gives oxygen or hydrogen gas. Whereas proton and oxygen ion spontaneously produce water, production of oxygen and hydrogen from water is nonspontaneous and should not occur. For this, the voltage between the electrode, which is exposed to the water and ions in the human body, and bodily fluid should not exceed a predetermined value. The voltage value means a threshold voltage value at which the reactions of Reaction Formulas 1 and 2 occur in the bodily fluid. The voltage may be a voltage applied between both ends of the capacitor C_{d1}, in the circuitry modeled as will be described later.

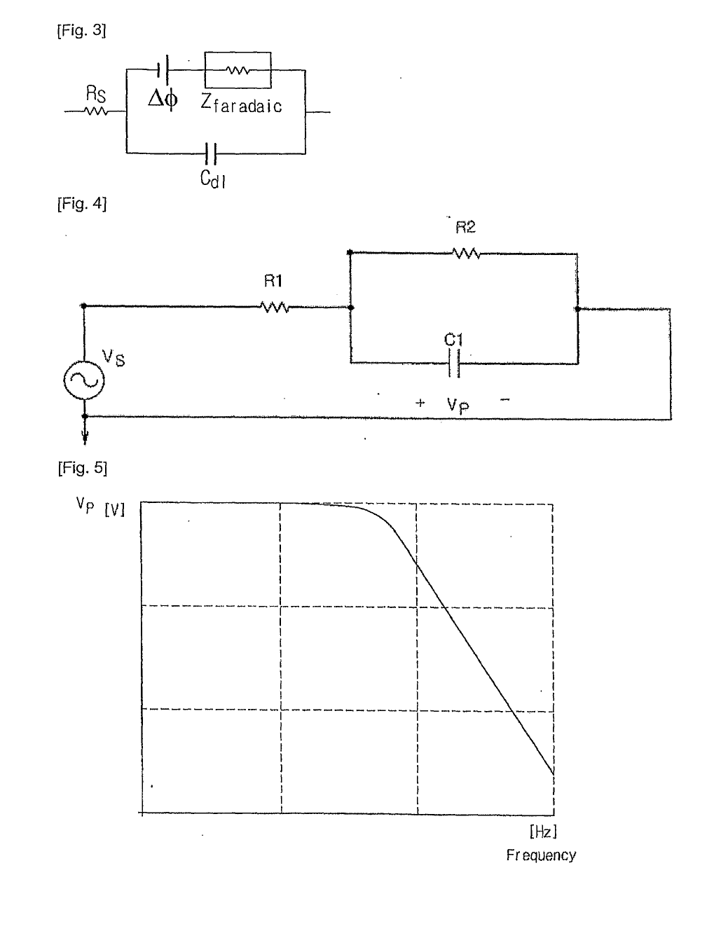

FIG. 3 is a circuit diagram of a double layer modeled as an electrical circuitry. Since the double layer behaves like a capacitance, it may be modeled as a capacitor C_{d1}. In FIG. 3, Δφ stands for equilibrium interfacial potential, Rₛ for fluid resistance, and Z_{faradaic} for faradaic impedance.

FIG. 4 is a circuit diagram illustrating a simulation of the double layer modeled as an electrical circuitry in FIG. 3. Suppose that a voltage V_{S} is applied between the electrodes of a neural stimulator for neural prosthesis exposed to bodily fluid. This means that power is leaking from the neural stimulator for neural prosthesis.

Suppose that a voltage V_{P} is applied at the double layer C_{d1}, in the model circuitry of FIG. 4. The above-mentioned irreversible reaction does not occur only when the voltage V_{P} is below a reference value. A simulation was carried out by varying frequencies, while keeping the voltage applied to the circuitry of FIG. 4 constant.

FIG. 5 shows frequency response of the voltage V_{P} applied between both ends of the capacitor in the equivalent circuit of FIG. 4. The graph exhibits a low pass filter (LPF) frequency response.

Referring to FIG. 5, when the frequency approaches 0 (i.e., a DC voltage) the voltage V_{P} is equal to the voltage V_{S} applied to the circuitry. This means that leakage of DC voltage in the body is highly likely to result in an irreversible reaction. The voltage V_{P} decreases as the frequency increases (i.e., an AC voltage). This means that leakage of AC voltage in the body is relatively less likely to result in an irreversible reaction than the DC voltage. Along with FIG. 2, this supports the safety of the neural stimulator for neural prosthesis using AC logic power.

In another embodiment according to this disclosure, AC logic power is supplied by a power converter in order to cope with current leakage, even when a neural stimulator is packaged by a complete hermetic packaging. The casing is made of titanium or ceramics.

A neural stimulator for neural prosthesis according to this embodiment comprises: a power receiver 11 which receives power from outside and supplies the received power to a circuitry including an electrical signal generator; the electrical signal generator 12 which generates a neural stimulating electrical signal; and a casing 13 which protects the power receiver and the electrical signal generator from bodily fluid, wherein the power receiver supplies AC logic power to the circuitry including the electrical signal generator. The casing may be a hermetic packaging using titanium or ceramics.

Leakage of electrical current may occur even when a casing for protecting power receiver and the electrical signal generator from bodily fluids or ions is used. In that case, use of DC power may be fatal to nearby tissues or cells as the monophasic stimulation by the DC power is accumulated at the tissues or cells. Therefore, AC logic power may be used as power source to ensure safety of the tissues or cells.

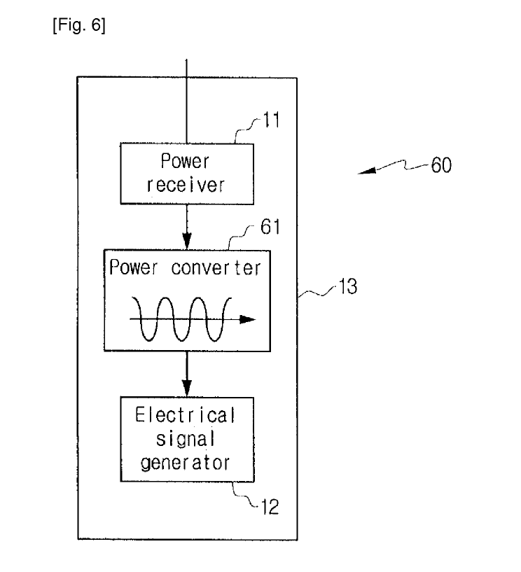

FIG. 6 is a schematic diagram illustrating a neural stimulator for neural prosthesis according to another embodiment to which a power converter is equipped.

Referring to FIG. 6, the neural stimulator for neural prosthesis comprises: a power receiver 11 which receives power from outside and supplies the received power to a power converter; an electrical signal generator 12 which generates a neural stimulating electrical signal; the power converter 61 which converts the power received from the power receiver into stable AC logic power and supplies stable AC logic power to a circuitry including the electrical signal generator; and a casing 13 which protects the power receiver, the electrical signal generator and the power converter from bodily fluid, wherein the power supplied by the power converter to the circuitry including the electrical signal generator is AC logic power. As described above, the casing may be a hermetic packaging using titanium or ceramics, or the casing may be coated with silicone or polyurethane the outside of the circuitry.

Hereinafter, it will be given a more detailed description of the embodiment of FIG. 6.

In accordance with this embodiment, the power received by the power receiver may be converted into AC logic power, which is more stable and suitable for the circuitry, and then supplied to the internal circuitry of the neural stimulator for neural prosthesis.

The neural stimulator for neural prosthesis according to the embodiment comprises: a power receiver which receives power from outside and supplies the received power to a power converter; an electrical signal generator which generates a neural stimulating electrical signal; the power converter which converts the power received from the power receiver into stable AC logic power and supplies stable AC logic power to a circuitry including the electrical signal generator; and a casing which protects the power receiver, the electrical signal generator and the power converter from bodily fluid, wherein the power supplied by the power converter to the circuitry including the electrical signal generator is AC logic power. As described above, the casing may be coated with silicone or polyurethane the outside of the power receiver, the electrical signal generator and the power converter.

The AC logic power may be, for example, one of a sine wave, a pulse wave and a triangular wave. The addition of the power converter may vary the electrical signal generators that may be used in the neural stimulator for neural prosthesis. That is to say, a variety of power sources may be used to operate the electrical signal generator.

In another embodiment according to this disclosure, a neural stimulator for neural prosthesis comprises a power converter which converts the power received by the power receiver into AC logic power, which is more stable and suitable for the circuitry, and then supplies AC logic power to the internal circuitry of the neural stimulator for neural prosthesis, when a hermetic packaging is used.

The neural stimulator for neural prosthesis according to the embodiment comprises: a power receiver which receives power from outside and supplies the received power to a power converter; an electrical signal generator which generates a neural stimulating electrical signal; the power converter which converts the power received from the power receiver into stable AC logic power and supplies stable AC logic power to a circuitry including the electrical signal generator; and a casing which protects the power receiver, the electrical signal generator and the power converter from bodily fluid, wherein the power supplied by the power converter to the circuitry including the electrical signal generator is AC logic power. The casing may be a hermetic packaging using titanium or ceramics.

As described above, the AC logic power may be, for example, one of a sine wave, a pulse wave and a triangular wave. The addition of the power converter may vary the electrical signal generators that may be used in the neural stimulator for neural prosthesis. That is to say, a variety of power sources may be used to operate the electrical signal generator.

The power receiver may receive the power used by the device from outside via wireless communication. In that case, the power may be received via wireless communication and induction of current through a coil.

Further, the power receiver may receive power from outside via photoinduced energy transfer.

In another embodiment, the power receiver may receive power from a battery which supplies DC power. The received DC power is converted into AC logic power by the power converter and then supplied to the electrical signal generator.

Like in the aforesaid other embodiments, safety and reliability of the electrical signal generator may be improved by converting the power used by the electrical signal generator into the AC logic power.

The existing neural prosthetic systems using DC power require that the entire system including the battery and the electrical signal generator be hermetically packaged using titanium or ceramics. Further, all the positions connected by the electrode are exposed to the risk of leakage of bodily fluid.

In contrast, in accordance with this disclosure, the electrical signal generator may be simply coated with a circuitry protecting material, and the positions exposed to the risk of leakage are just the two connection positions of the hermetic battery and the power converter for power supply.

Further, since the battery occupying a large volume is separable from the electrical signal generator, the battery may be disposed freely as separated from the electrical signal generator.

While the exemplary embodiments have been shown and described, it will be understood by those skilled in the art that various changes in form and details may be made thereto without departing from the spirit and scope of this disclosure as defined by the appended claims.

In addition, many modifications can be made to adapt a particular situation or material to the teachings of this disclosure without departing from the essential scope thereof. Therefore, it is intended that this disclosure not be limited to the particular exemplary embodiments disclosed as the best mode contemplated for carrying out this disclosure, but that this disclosure will include all embodiments falling within the scope of the appended claims.

1. A neural stimulator for neural prosthesis comprising:
a power receiver which receives power from outside and supplies the received power to a circuitry including an electrical signal generator;
the electrical signal generator which generates a neural stimulating electrical signal; and
a casing which protects the power receiver and the electrical signal generator from bodily fluid,
wherein the power supplied by the power receiver to the circuitry including the electrical signal generator is AC logic power.

2. The neural stimulator for neural prosthesis according to claim 1, wherein the casing is a hermetic packaging using titanium or ceramics.

3. The neural stimulator for neural prosthesis according to claim 1, wherein the casing coats with a circuitry protecting material the outside of the power receiver and the electrical signal generator.

4. The neural stimulator for neural prosthesis according to claim 1, wherein the power receiver receives power from outside via wireless communication or photoinduced energy transfer.

5. The neural stimulator for neural prosthesis according to claim 3, wherein the circuitry protecting material comprises an insulating material.

6. The neural stimulator for neural prosthesis according to claim 5, wherein the circuitry protecting material comprises silicone elastomer or polyurethane.

7. A neural stimulator for neural prosthesis comprising:
a power receiver which receives power from outside and supplies the received power to a circuitry including a power converter;
an electrical signal generator which generates a neural stimulating electrical signal;
a power converter which converts the power received from the power receiver into stable AC logic power and supplies it to the electrical signal generator; and
a casing which protects the power receiver, the electrical signal generator and the power converter from bodily fluid,
wherein the power supplied by the power converter to a circuitry including the electrical signal generator is AC logic power.

8. The neural stimulator for neural prosthesis according to claim 7, wherein the casing is a hermetic packaging using titanium or ceramics.

9. The neural stimulator for neural prosthesis according to claim 7, wherein the casing coats with a circuitry protecting material the outside of the power receiver, the electrical signal generator and the power converter.

10. The neural stimulator for neural prosthesis according to claim 7, wherein the AC logic power is any one of a sine wave, a pulse wave and a triangular wave.

11. The neural stimulator for neural prosthesis according to claim 7, wherein the power receiver receives power from outside via wireless communication or photoinduced energy transfer.

12. The neural stimulator for neural prosthesis according to claim 9, wherein the circuitry protecting material comprises an insulating material.

13. The neural stimulator for neural prosthesis according to claim 12, wherein the circuitry protecting material comprises silicone elastomer or polyurethane.

14. The neural stimulator for neural prosthesis according to claim 7, wherein the power receiver receives power from a battery which supplies DC power.
